(19) United States
(12) Patent Application Publication
(10) Pub. No.: US 2018/0085303 A1
(43) Pub. Date: Mar. 29, 2018

(54) COSMETIC COMPOSITIONS COMPRISING FOOD-GRADE COMPONENTS AND METHODS OF MAKING AND USING SAME

(71) Applicant: Smoothie Beauty, Inc., New York, NY (US)

(72) Inventor: Stephanie Michelle Peterson, New York, NY (US)

(73) Assignee: Smoothie Beauty, Inc.

(21) Appl. No.: 15/713,093

(22) Filed: Sep. 22, 2017

Related U.S. Application Data
(60) Provisional application No. 62/398,805, filed on Sep. 23, 2016.

(51) Int. Cl. A61K 8/98; A61K 8/19; A61K 8/9789; A61K 8/9794; A61K 8/99; A61K 8/9711; A61Q 19/00; A61Q 19/08; A61L 2/02; A45D 40/00; B65B 7/02; B65B 3/04; B65B 55/04; B65D 85/00

(52) U.S. Cl. CPC A61K 8/988; A61K 2800/882; A61K 8/9789; A61K 8/9794; A61K 8/99; A61K 8/986; A61K 8/9711; A61Q 19/007; A61Q 19/08; A61Q 19/00; A61L 2/02; A45D 40/00; B65B 7/02; B65B 3/04; B65B 55/04; B65D 85/70; A61K 2800/85; A61K 2800/87; A61K 8/19

(57) ABSTRACT

The present disclosure provides sterilized, shelf-stable cosmetic compositions comprising food-grade components and no preservatives, and methods of making and using same.

### TECHNICAL FIELD

The present disclosure relates to cosmetic compositions comprising, consisting essentially of, or consisting of food-grade components, and methods of making and using same.

### BACKGROUND

Widely distributed cosmetic compositions typically include one or more preservatives or other excipients in order to, for example, discourage microbial growth. However, the preservatives or other excipients may damage skin, or may reduce beneficial properties of other components of the cosmetic composition. Cosmetic compositions that include biodegradable components, such as food-grade components, are especially susceptible to microbial growth and thus regularly include preservatives, such as antimicrobial compounds.

Currently, cosmetic compositions including food-grade components without also including preservatives are not commercially available. A need exists for such cosmetic compositions that have a reasonable shelf-life (e.g., no or minimal microbial growth after about 30 days) without including preservatives.

### SUMMARY

The present disclosure provides sterilized, shelf-stable cosmetic compositions comprising food-grade components and no preservatives, and methods of making and using same.

In some embodiments, the present disclosure provides a cosmetic composition comprising at least one food-grade component, but no preservative.

In some embodiments, the present disclosure provides a cosmetic composition consisting essentially of at least one food-grade component, but no preservative.

In some embodiments, the present disclosure provides a cosmetic composition consisting of at least one food-grade component, but no preservative.

In some embodiments, the present disclosure provides a packaged cosmetic product comprising a sterilized cosmetic composition comprising, consisting essentially of, or consisting of at least one food-grade product, wherein the sterilized cosmetic composition does not include a preservative.

In some embodiments, the present disclosure provides a kit comprising, in a first pouch, a packaged cosmetic product comprising a sterilized cosmetic composition comprising, consisting essentially of, or consisting of at least one food-grade product; and, in a second pouch, at least one enhancing agent, optionally selected from matcha powder, charcoal powder, maca powder, and combinations thereof.

In some embodiments, the present disclosure provides a method of making a sterilized cosmetic product, the method comprising: combining a fruit juice, a honey, turmeric root, a fruit peel, yogurt and flour to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining cacao powder, coffee granules, maple syrup, a vegetable oil, and almond milk to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining a fruit juice, a honey, a vegetable oil, coconut water, and a fruit puree to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining a fruit juice, a honey, a vegetable oil, yogurt and vanilla bean powder to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining a honey, a fruit pulp, a fruit juice, and a fruit puree to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining a fruit pulp, a succulent puree, yogurt and kelp granules to form a cosmetic composition; placing at least a portion of the cosmetic composition in a pouch; sealing the pouch; and sterilizing the sealed pouch to form a sterilized cosmetic product.

In some embodiments, the present disclosure provides a method of making a sterilized cosmetic product, the method comprising: combining a honey, at least two fruit purees, and mint to form a cosmetic composition; placing at least a portion of the cosmetic composition in a pouch; sealing the pouch; and sterilizing the sealed pouch to form a sterilized cosmetic product.

In some embodiments, the present disclosure provides a method of using a cosmetic composition, the method comprising: combining a sterilized cosmetic composition with an additive to form a ready-to-use cosmetic composition; and applying the ready-to-use cosmetic composition to skin.

These and other embodiments are described in further detail below.

### BRIEF DESCRIPTION OF THE DRAWINGS

FIG. 1 shows a sterilized packaged cosmetic product consistent with the present disclosure. The sterilized packaged cosmetic product comprises a pouch and a sterilized cosmetic composition housed within the pouch.

FIG. 2 shows a kit comprising a sterilized packaged cosmetic product, an enhancing agent, and an optional funnel.

### DETAILED DESCRIPTION

The present disclosure provides cosmetic products comprising a cosmetic composition and an enhancing agent, wherein the cosmetic composition does not include a preservative. The present disclosure also provides packaged cosmetic products and kits comprising a cosmetic composition and an enhancing agent, wherein the cosmetic composition does not include a preservative.

### 1. Cosmetic Compositions

The present disclosure provides cosmetic compositions comprising, consisting essentially of, or consisting of food-grade components, such as one or more of: fruit juices, fruit purees, honeys, herbs, fruit peels and/or zests, yogurts, flours, vegetable oils, syrups, almond milk, cacao powder, coffee, vanilla bean powder, coconut water, fruit pulp, kelp, and/or mint; and one or more optional enhancing agents. The cosmetic compositions do not include a preservative. In some embodiments, the cosmetic composition does not include any excipient (other than a food-grade component).

Cosmetic compositions according to the present disclosure comprise at least one food-grade component, such as one or more of: fruit juices, fruit purees, honeys, herbs, fruit peels and/or zests, yogurts, flours, vegetable oils, syrups, almond milk, cacao powder, coffee, vanilla bean powder, coconut water, fruit pulp, kelp, and/or mint. In some embodiment, the cosmetic composition consists essentially of the at least one food-grade component. In some embodiments, the cosmetic composition consists of the at least one food-grade component.

In some embodiments, the cosmetic composition does not include added preservative beyond those naturally occurring in plants and vegetables without refinement or concentrated processing. Non-limiting examples of preservatives that are excluded from the cosmetic compositions of such embodiments include benzoic acid, propionic acid, salicylic acid, sorbic acid, biphenyl-2-ol, 4-hydroxybenzoic acid, 3-acetyl-6-methylpyran-2,4-(3H)-dione, formic acid, 3-3′-dibromo-4,4′-hexamethyulenedioxydibenzamidineundec-10-enoic acid, 1,6-di-(4-amidinophenoxy)-n-hexane, cosmetically acceptable salts and/or esters of the foregoing, phenoxyethanol, ethylhexylglycerine, a mixture of phenoxyethanol and ethylhexylglycerine (EUXYL PE 9010), zinc pyrithione, inorganic sulphites, hydrogensulphites, chlorobutanol, thiomersal, phenylmercuric salts, hexetidine, 5-bromo-5-nitro-1,3-dioxane, bronopol, 2,4-dichlorobenzyl alcohol, triclocarban, 4-chloro-m-cresol, triclosan, 4-chloro-3,5-xylenol, 3,3′-bis-(1-hydroxymethyl-2,5-dioxoimidazolidin-4-yl)-1,1′-methylenediurea (also referred to as imidazolidinyl urea), poly-(1-hexamethylenebiguanide hydrochloride), 2-phenoxyethanol, hexamethylenetetramine (methenamine), methenamine 3-chloroallylochloride, 1-(4-chlorophenoxy)-1-(imidazol-1-yl)-3,3-dimethylbutan-2-one, 1,3-bis(hydroxymethyl)-5,5-dimethylimidazoline-2,4-dione, benzyl alcohol, 1-hydroxy-4-methyl-6-(2,4,4-trimethylpentyl)-2-pyridon, monoethanolamine salt of 1-hydroxy-4-methyl-6-(2,4,4-trimethylpentyl)-2-pyridon, 6,6-dibromo-4,4-dichloro-2,2′-methylenediphenol:bromochlorophen, 4-isopropyl-m-cresol, a mixture of 5-chloro-2-methylisothiazol-3-(2H)-one and 2-methylisothizaol-3-(2H)-one with magnesium chloride and magnesium nitrate, 2-benzyl-4-chlorophenol (also referred to as chlorophene), chlorhexidine and its digluconate, diacetate and dihydrochloride, 1-phenoxypropan-2-ol, alkyl (C12-22) trimethyl ammonium bromide and chloride, 4,4-dimethyl-1,3-oxazolidine, N-(hydroxymethyl)-N-(dihydroxymethyl-1,3-dioxo-2,5-imidazolinidyl-4)-N′-(hydroxymethyl) urea, glutaraldehyde, 5-ethyl-3,7-dioxa-1-azabicyclo[3.3.0]octane, 3-(p-chlorophenoxy)-propane-1,2-diol (also referred to as chlorphenesin), sodium hydroxymethylamino acetate, silver chloride deposited on titanium dioxide, benzethionium chloride, benzalkonium chloride, benzalkonium bromide, benzalkonium saccharinate, benzylhemiformal, iodopropynyl butyl-carbamate, methylisothiazolidinone, and combinations thereof.

In some embodiments, the cosmetic composition does not include an added fragrance.

In some embodiments, the cosmetic composition does not include an antimicrobial agent. Non-limiting examples of antimicrobial agents that are excluded from cosmetic compositions of such embodiments include salicylic acid, azelaic acid, furosemide, benzoyl peroxide and neomycin.

In some embodiments, the cosmetic composition does not include a sequestrant. Non-limiting examples of sequestrants that are excluded from cosmetic compositions of such embodiments include tetrasodium EDTA (TRILON B), disodium EDTA (TRILON B), calcium disodium EDTA, glucono deltalactone, sodium gluconate, potassium gluconate, sodium tripolyphosphate, sodium hexametaphosphate, and combinations thereof.

In some embodiments, the cosmetic composition does not include a synthetic humectant. Non-limiting examples of synthetic humectants that are excluded from cosmetic compositions of such embodiments include propylene glycol, glyceryl triacetate, vinyl alcohol, neoagarobiose, a sugar polyol, a polymeric polyol, quillaia, synthetic or refined lactic acid, urea, non-food grade glycerine, MP Diol, synthetic or refined alpha-hydroxy acid, and combinations thereof.

In some embodiments, the cosmetic composition does not include a thickening agent. Non-limiting examples of thickening agents that are excluded from cosmetic compositions of such embodiments include sodium polyacrylate and polymeric sulfonic acid.

In some embodiments, the cosmetic composition does not include a synthetic emulsifier. Non-limiting examples of emulsifiers that are excluded from cosmetic compositions of such embodiments include glyceryl stearate citrate, glyceryl stearate, behenyl alcohol, and combinations thereof.

In some embodiments, the cosmetic composition does not include a synthetic emollient. Non-limiting examples of emollients that that are excluded from cosmetic compositions of such embodiments include propylheptyl caprylate, C₁₂-C₁₅ alkyl benzoate, cetearyl isononanoate, butyrospermum parkii butter, and combinations thereof.

In some embodiments, the cosmetic composition does not include a synthetic adsorbing agent. One non-limiting example of an adsorbing agent that is excluded from cosmetic compositions of such embodiments is aluminum starch octenylsuccinate.

In some embodiments, the cosmetic composition does not include a synthetic antioxidant. Non-limiting examples of synthetic emollients that that are excluded from cosmetic compositions of such embodiments include dimethylmethoxy chromanol; tocopherol; syntheteic or refined lecithin; ascorbyl palmitate; synthetic or refined citric acid; a mixture of tocopherol, lecithin, ascorbyl palmitate, and citric acid; idebenone; and combinations thereof.

In some embodiments, the cosmetic composition does not include a pH modifier. One non-limiting example of a pH modifier that is excluded from cosmetic compositions of such embodiments is sodium hydroxide.

In some embodiments, the cosmetic composition does not include a texturizing agent. Non-limiting examples of texturizing agents that are excluded from cosmetic compositions of such embodiments include PEG-8-dimethicone, polysilicone-11, laureth-12, a crosspolymer of dimethicone and detearyl dimethicone, silica, polyurethane, polymethylsilsesquioxane, a mixture of polysilicone-11 and laureth-12, a mixture of polyurethane and polymethylsilsesquioxane, and combinations thereof.

In some embodiments, the cosmetic composition does not include any synthetic sequestrants, humectants, thickening agents, emulsifiers, emollients, adsorbing agents, preservatives, fragrances, antioxidants, pH modifiers, or texturizing agents, other than those naturally present in the food-grade components.

In some embodiments, the cosmetic composition comprises a fruit juice. The fruit juice comprises lemon juice. In some embodiments, the fruit juice comprises, consists essentially of, or consists of lime juice. In some embodiments, the fruit juice comprises, consists essentially of, or consists of pineapple juice. In some embodiments, the fruit juice comprises, consists essentially of, or consists of papaya juice.

In some embodiments, the cosmetic composition comprises citrus peel. In some embodiments, the citrus peel comprises lemon peel or lemon zest.

In some embodiments, the cosmetic composition comprises fruit pulp. In some embodiments, the fruit pulp comprises, consists essentially of, or consists of pineapple pulp. In some embodiments, the fruit pulp comprises, consists essentially of, or consists of papaya pulp. In some embodiments, the fruit pulp comprises, consists essentially of, or consists of cucumber pulp.

In some embodiments, the cosmetic composition comprises fruit puree. In some embodiments, the fruit puree comprises, consists essentially of, or consists of avocado puree. In some embodiments, the fruit puree comprises, consists essentially of, or consists of banana puree. In some embodiments, the fruit puree comprises, consists essentially of, or consists of kiwi puree. In some embodiments, the fruit puree comprises, consists essentially of, or consists of blackberry puree.

In some embodiments, the cosmetic composition comprises a succulent puree. In some embodiments, the succulent puree comprises, consists essentially of, or consists of aloe vera puree.

In some embodiments, the cosmetic composition comprises honey. In some embodiments, the honey comprises, consists essentially of, or consists of Manuka honey. In some embodiments, the honey comprises, consists essentially of, or consists of raw honey. In some embodiments, the honey comprises, consists essentially of, or consists of raw neem honey.

In some embodiments, the cosmetic composition comprises turmeric root.

In some embodiments, the cosmetic composition comprises yogurt. In some embodiments, the yogurt comprises, consists essentially of, or consists of whole milk Greek yogurt.

In some embodiments, the cosmetic composition comprises flour. In some embodiments, the flour comprises, consists essentially of, or consists of whole oat flour.

In some embodiments, the cosmetic composition comprises cacao powder. In some embodiments, the cacao powder comprises, consists essentially of, or consists of raw cacao powder.

In some embodiments, the cosmetic composition comprises coffee. In some embodiments, the coffee comprises, consists essentially of, or consists of coffee in granular form.

In some embodiments, the cosmetic composition comprises maple syrup.

In some embodiments, the cosmetic composition comprises a vegetable oil. In some embodiments, the vegetable oil comprises, consists essentially of, or consists of coconut oil. In some embodiments, the coconut oil is extra virgin unrefined cold-pressed coconut oil. In some embodiments, the vegetable oil comprises, consists essentially of, or consists of avocado oil. In some embodiments, the avocado oil is unrefined cold-pressed avocado oil. In some embodiments, the vegetable oil comprises, consists essentially of, or consists of olive oil. In some embodiments, the olive oil is extra virgin unrefined cold-pressed olive oil.

In some embodiments, the cosmetic composition comprises almond milk.

In some embodiments, the cosmetic composition comprises coconut water. In some embodiments, the coconut water comprises, consists essentially of, or consists of raw coconut water.

In some embodiments, the cosmetic composition comprises vanilla bean powder.

In some embodiments, the cosmetic composition comprises kelp granules.

In some embodiments, the cosmetic composition comprises mint. In some embodiments, the mint is in leaf form.

In some embodiments, the cosmetic composition comprises, consists essentially of, or consists of a fruit juice, a honey, turmeric root, a fruit peel, yogurt and flour. In some embodiments, the fruit juice is lemon juice, the honey is Manuka honey, the fruit peel is lemon peel, the yogurt is whole milk Greek yogurt, and the flour is whole oat flour. Such cosmetic compositions may be especially useful for a skin brightening cosmetic product, such as a facial mask.

In some embodiments, the cosmetic composition comprises, consists essentially of, or consists of cacao powder, coffee granules, maple syrup, a vegetable oil, and almond milk. In some embodiments, the cacao powder is raw cacao powder, the coffee is in granular form, the maple syrup is dark maple syrup, and the vegetable oil is extra virgin unrefined cold-pressed coconut oil. Such cosmetic compositions may be especially useful for a stimulating cosmetic product, such as a facial mask.

In some embodiments, the cosmetic composition comprises, consists essentially of, or consists of a fruit juice, a honey, a vegetable oil, coconut water, and a fruit puree. In some embodiments, the fruit juice is lime juice, the honey is raw honey, the vegetable oil is unrefined cold-pressed avocado oil, the coconut water is raw coconut water, and the fruit puree is avocado puree. Such cosmetic compositions may be especially useful for consumers with dry skin, such as facial masks for consumers with dry skin.

In some embodiments, the cosmetic composition comprises, consists essentially of, or consists of a fruit juice, a honey, a vegetable oil, yogurt and vanilla bean powder. In some embodiments, the fruit juice is lemon juice, the honey is Manuka honey, the vegetable oil is extra virgin unrefined cold-pressed olive oil, and the yogurt is whole milk Greek yogurt. Such cosmetic compositions may be especially useful for anti-aging cosmetic products, such as facial masks.

In some embodiments, the cosmetic composition comprises, consists essentially of, or consists of a honey, a fruit pulp, a fruit juice, and a fruit puree. In some embodiments, the honey is raw honey, the fruit pulp comprises pineapple pulp and papaya pulp, and the fruit puree is banana puree. Such cosmetic compositions may be especially useful for consumers with oily skin, such as facial masks for consumers with oily skin.

In some embodiments, the cosmetic composition comprises, consists essentially of, or consists of a fruit pulp, a succulent puree, yogurt and kelp granules. In some embodiments, the fruit pulp is cucumber pulp, the succulent puree is aloe vera puree, and the yogurt is whole milk Greek yogurt. Such cosmetic compositions may be especially useful for soothing cosmetic products, such as facial masks.

In some embodiments, the cosmetic composition comprises, consists essentially of, or consists of a honey, at least two fruit purees, and mint. In some embodiments, the honey is raw neem honey, the at least two fruit purees comprise kiwi puree, blackberry puree, and banana puree, and the mint is in leaf form. Such cosmetic compositions may be especially useful for a revitalizing cosmetic product, such as a facial mask.

### 2. Enhancing Agents

Cosmetic products (e.g., packaged cosmetic products or kits) of the present disclosure may also include one or more enhancing agents. In some embodiments, the enhancing agent is present as a component of the cosmetic composition. In other embodiments, the enhancing agent is provided to the consumer in a form separate from the cosmetic composition, and may be added to the cosmetic composition by the consumer, for example before applying the cosmetic composition (e.g., immediately before applying the cosmetic composition) to skin. In some embodiments, about 0.5 teaspoons of the enhancing agent is added for every 40 mL of the cosmetic product.

In some embodiments, the cosmetic product (e.g., a packaged cosmetic product or a kit) comprises an enhancing agent selected from the group consisting of matcha powder, charcoal powder (e.g., activated charcoal powder), and macha powder.

In some embodiments, the enhancing agent comprises, consists essentially of, or consists of matcha powder. Such products may be especially useful in anti-aging cosmetic products, such as facial masks.

In some embodiments, the enhancing agent comprises, consists essentially of, or consists of charcoal powder (e.g., activated charcoal powder). Such products may be especially useful in detoxifying cosmetic products, such as facial masks.

In some embodiments, the enhancing agent comprises, consists essentially of, or consists of macha powder. Such products may be especially useful in energizing cosmetic products, such as facial masks.

### 3. High Pressure Processing Sterilization.

High Pressure Processing is a cold pasteurization technique which consists of subjecting food, previously sealed in flexible and water-resistant packaging, to a high level of hydrostatic pressure (pressure transmitted by water) up to 600 MPa/87,000 psi or more for a few seconds to a few minutes.

### 4. Packaged Cosmetic Products and Kits

The present disclosure also provides packaged cosmetic products. In general, a packaged cosmetic product comprises a cosmetic composition as disclosed herein in a container, such as a pouch. In some embodiments, the packaged cosmetic product comprises a sterilized cosmetic composition in a container, such as a pouch. The cosmetic composition may be sterilized before being placed in the container (e.g., pouch). In some embodiments, the cosmetic composition is sterilized after being placed and sealed in the container (e.g., the cosmetic composition is sterilized after being sealed in a pouch). In such embodiments, the cosmetic composition may be sterilized by high pressure processing, for example by exposing the pouch comprising the cosmetic composition to a pressure of up to about 600 MPa (up to about 87,000 psi) for a period of time of about 1 second to about 5 minutes. In some embodiments, a sealed pouch comprising the cosmetic composition is exposed to a pressure of about 600 MPa (about 87,000 psi) for about 3 minutes (i.e., 180 seconds).

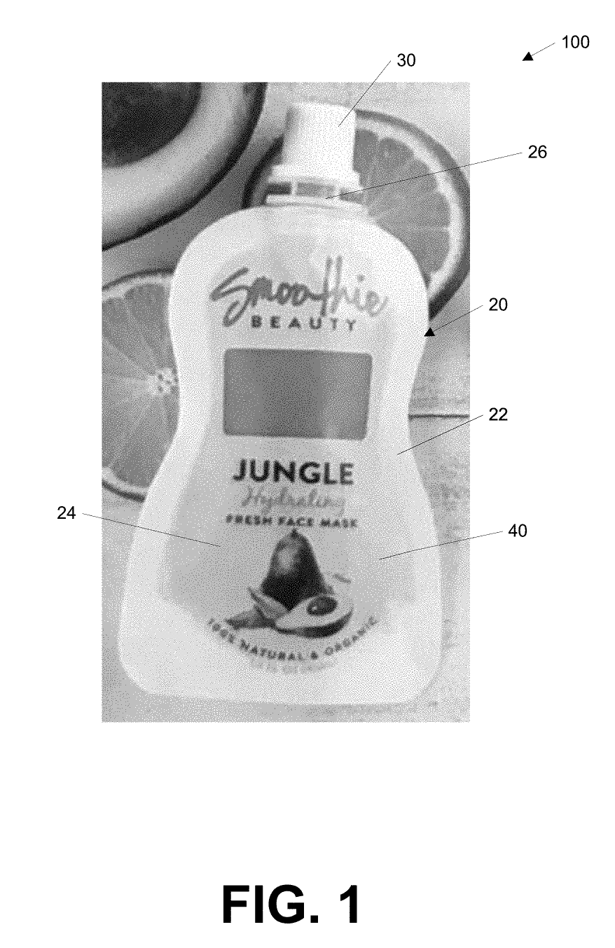

Referring now to FIG. 1, the present disclosure provides a sterilized packaged cosmetic product 100 comprising a pouch 20, a seal 30, and a cosmetic composition 40 housed within the pouch 20. The pouch 20 may be formed from two sheets of material (e.g., food-grade material) or from a single sheet folded on itself. A sealed edge portion 22 binds the two sheets (or folded single sheet) together around the periphery of the pouch 20, and defines a cavity 24 internal to the pouch 20. The pouch 20 further includes a seal portion 26 which, in the embodiment shown in FIG. 1, mates with a cap 30 to seal the cosmetic composition 40 inside the pouch 20.

In some embodiments, the cosmetic composition 40 is inserted into the cavity 24 of the pouch 20, the cap 30 is secured to the seal portion 26, and the combination is then subjected to high pressure processing as described below to form the sterilized packaged cosmetic product 100.

In some embodiments, the pouch 20 (e.g., the material forming the pouch 20) consists essentially or consists of a food grade material.

In some embodiments, the pouch 20 includes a cavity 24 capable of holding about 5 mL to about 100 mL of the cosmetic composition 40, for example about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL of the cosmetic composition 40. In some embodiments, the cavity 24 is capable of holding about 40 mL of the cosmetic composition 40.

In some embodiments, the pouch 20 is capable of withstanding high pressure processing when in a sealed configuration. In some embodiments, the high pressure processing comprises applying a pressure of up to about 600 MPa (about 87,000 psi) for up to about 5 minutes. Various pressure and time periods may be used to apply pressure to the pouch 20 and cosmetic composition 40. For example, a pressure of about 400 MPa may be applied for between 1 and 10 minutes, a pressure of 450 MPa may be applied for between 1 and 10 minutes, a pressure of 500 MPa may be applied between 1 and 10 minutes, a pressure of 550 MPa may be applied between 1 and 10 minutes, a pressure of 600 MPa may be applied for between 1 and 10 minutes, a pressure of 650 MPa may be applied between 1 and 10 minutes. A pressure of 700 MPa or more may be applied between 1 and 10 minutes. Various pressures may be applied for between less than 1 minute to over 60 minutes. A dynamic pressure gradient may be applied over a time period, for example, a high pressure of between 300 MPa to 600 MPa may be applied for a first period of time and a higher pressure of between 350 MPa to 700 MPa may be applied for a second period of time. In other embodiments, a dynamic pressure gradient may be applied over a time period, for example, a higher pressure of between 350 MPa to 700 MPa may be applied for a first period of time and a high pressure of between 300 MPa to 650 MPa may be applied for a second period of time. Pressure may be increasingly applied over a period of time and pressure may be relieved or decreased over a period of time.

In some embodiments, the sterilized packaged cosmetic product 100 is shelf-stable for at least 30 days, for example at least 30 days, at least about 35 days, at least about 40 days, at least about 45 days, at least about 50 days, at least about 55 days, at least about 60 days, at least about 65 days, at least about 70 days, at least about 75 days, at least about 80 days, at least about 85 days, at least about 90 days, at least about 95 days, at least about 100 days, when stored (e.g., stored in a sealed configuration) at about 5° C.

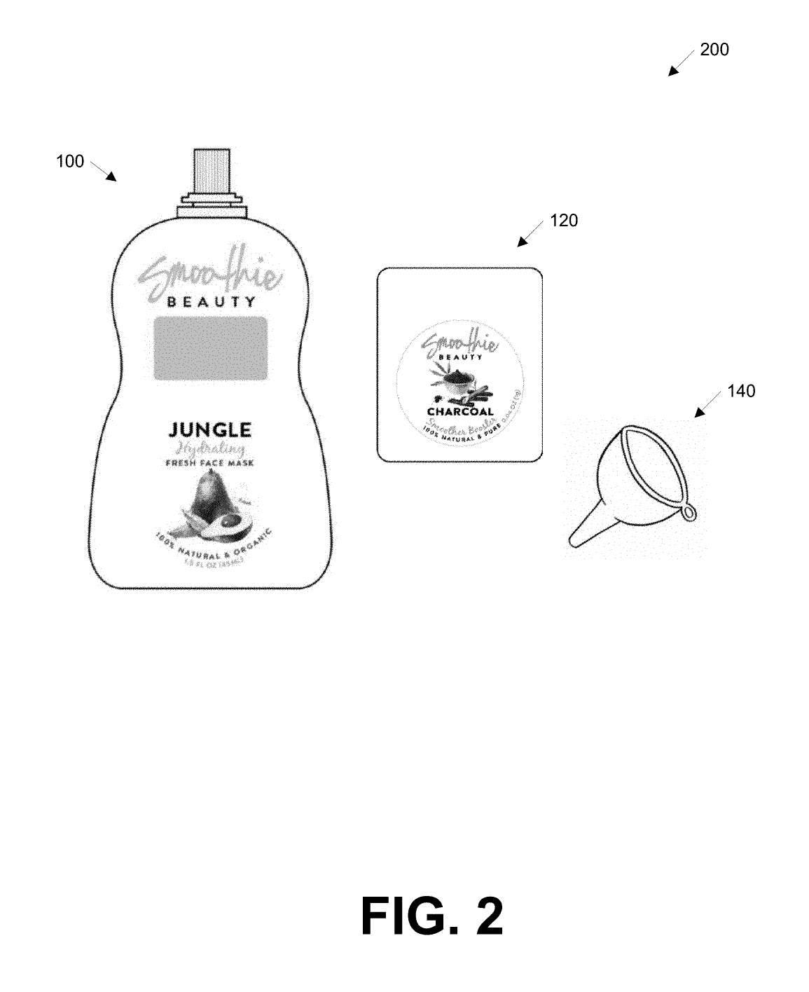

Referring now to FIG. 2, the present disclosure also provides a kit 200 comprising a sterilized packaged cosmetic product 100 as disclosed herein in a first pouch 20, and an enhancing agent in a second pouch 120. In some embodiments, the enhancing agent is at least one of: matcha powder, charcoal powder and maca powder.

In some embodiments, the kit 200 further comprises a funnel 140 for adding the enhancing agent to the first pouch 20, for example to form a ready-to-use cosmetic composition.

In some embodiments, the enhancing agent is sterilized.

In some embodiments, the kit further comprises instructions for mixing the enhancing agent with the sterilized cosmetic composition to form a ready-to-use cosmetic composition, and applying the ready-to-use cosmetic composition to skin.

### 5. Methods of Making a Sterilized Cosmetic Product

The present disclosure also provides methods of making a sterilized cosmetic product. In general, the methods comprise preparing a cosmetic composition and thereafter sterilizing the cosmetic composition to form the sterilized cosmetic product. In some embodiments, the method further comprises placing the cosmetic composition in a pouch and sealing the pouch before the step of sterilizing, for example by high pressure processing. In general, such sterilized cosmetic products are shelf-stable (e.g., the sterilized cosmetic product is free or substantially free of live bacteria) for at least 30 days when stored at about 5° C. In some embodiments the sterilized cosmetic product is shelf-stable for at least 21 days when stored at between 5° C. and 15° C. In some embodiments the sterilized cosmetic products are shelf-stable for at least 5 days when stored at between 15° C. and 21° C.

In some embodiments, the present disclosure provides a method of making a sterilized cosmetic product, the method comprising combining a fruit juice, a honey, turmeric root, a fruit peel, yogurt and flour to form a cosmetic composition; placing at least a portion of the cosmetic composition in a pouch; sealing the pouch; and sterilizing the sealed pouch to form a sterilized cosmetic product. In some embodiments, the fruit juice is lemon juice, the honey is Manuka honey, the fruit peel is lemon peel, the yogurt is whole milk Greek yogurt, and the flour is whole oat flour. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining cacao powder, coffee granules, maple syrup, a vegetable oil, and almond milk to form a cosmetic composition; placing at least a portion of the cosmetic composition in a pouch; sealing the pouch; and sterilizing the sealed pouch to form a sterilized cosmetic product. In some embodiments, the cacao powder is raw cacao powder, the coffee is in granular form, the maple syrup is dark maple syrup, and the vegetable oil is extra virgin unrefined cold-pressed coconut oil. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining a fruit juice, a honey, a vegetable oil, coconut water, and a fruit puree to form a cosmetic composition; placing at least a portion of the cosmetic composition in a pouch; sealing the pouch; and sterilizing the sealed pouch to form a sterilized cosmetic product. In some embodiments, the fruit juice is lime juice, the honey is raw honey, the vegetable oil is unrefined cold-pressed avocado oil, the coconut water is raw coconut water, and the fruit puree is avocado puree. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining a fruit juice, a honey, a vegetable oil, yogurt and vanilla bean powder to form a cosmetic composition; placing at least a portion of the cosmetic composition in a pouch; sealing the pouch; and sterilizing the sealed pouch to form a sterilized cosmetic product. In some embodiments, the fruit juice is lemon juice, the honey is Manuka honey, the vegetable oil is extra virgin unrefined cold-pressed olive oil, and the yogurt is whole milk Greek yogurt. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining a honey, a fruit pulp, a fruit juice, and a fruit puree to form a cosmetic composition; placing at least a portion of the cosmetic composition in a pouch; sealing the pouch; and sterilizing the sealed pouch to form a sterilized cosmetic product. In some embodiments, the honey is raw honey, the fruit pulp comprises pineapple pulp and papaya pulp, and the fruit puree is banana puree. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining a fruit pulp, a succulent puree, yogurt and kelp granules to form a cosmetic composition; placing at least a portion of the cosmetic composition in a pouch; sealing the pouch; and sterilizing the sealed pouch to form a sterilized cosmetic product. In some embodiments, the fruit pulp is cucumber pulp, the succulent puree is aloe vera puree, and the yogurt is whole milk Greek yogurt. In some embodiments, the sterilized cosmetic product is shelf-stable (e.g., the sterilized cosmetic product is free or substantially free of live bacteria) for at least 30 days when stored at about 5° C.

In some embodiments, the present disclosure provides a method of making a sterilized cosmetic product, the method comprising combining a honey, at least two fruit purees, and mint to form a cosmetic composition; placing at least a portion of the cosmetic composition in a pouch; sealing the pouch; and sterilizing the sealed pouch to form a sterilized cosmetic product. In some embodiments, the honey is raw neem honey, the at least two fruit purees comprise kiwi puree, blackberry puree, and banana puree, and the mint is in leaf form. In some embodiments, the sterilized cosmetic product is shelf-stable (e.g., the sterilized cosmetic product is free or substantially free of live bacteria) for at least 30 days when stored at about 5° C.

### 6. Methods of Use

The present disclosure also provides methods of using cosmetic products (e.g., cosmetic compositions and optional enhancer(s)). In general, the methods of use comprise combining a sterilized cosmetic composition with the optional enhancing agent(s) to form a ready-to-use cosmetic composition; and thereafter applying the ready-to-use cosmetic composition to skin.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a fruit juice, a honey, turmeric root, a fruit peel, yogurt and flour. In some embodiments, the fruit juice is lemon juice, the honey is Manuka honey, the fruit peel is lemon peel, the yogurt is whole milk Greek yogurt, and the flour is whole oat flour. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of cacao powder, coffee granules, maple syrup, a vegetable oil, and almond milk. In some embodiments, the cacao powder is raw cacao powder, the coffee is in granular form, the maple syrup is dark maple syrup, and the vegetable oil is extra virgin unrefined cold-pressed coconut oil. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a fruit juice, a honey, a vegetable oil, coconut water, and a fruit puree. In some embodiments, the fruit juice is lime juice, the honey is raw honey, the vegetable oil is unrefined cold-pressed avocado oil, the coconut water is raw coconut water, and the fruit puree is avocado puree. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a fruit juice, a honey, a vegetable oil, yogurt and vanilla bean powder. In some embodiments, the fruit juice is lemon juice, the honey is Manuka honey, the vegetable oil is extra virgin unrefined cold-pressed olive oil, and the yogurt is whole milk Greek yogurt. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a honey, a fruit pulp, a fruit juice, and a fruit puree. In some embodiments, the honey is raw honey, the fruit pulp comprises pineapple pulp and papaya pulp, and the fruit puree is banana puree. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a fruit pulp, a succulent puree, yogurt and kelp granules. In some embodiments, the fruit pulp is cucumber pulp, the succulent puree is aloe vera puree, and the yogurt is whole milk Greek yogurt. In some embodiments, the enhancing agent is any one or more of: matcha powder, charcoal powder, and maca powder.

In some embodiments, a method of use according to the present disclosure comprises combining a sterilized cosmetic composition with an enhancing agent to form a ready-to-use cosmetic composition; and applying the ready-to-use cosmetic composition to skin, wherein the sterilized cosmetic composition comprises, consists essentially of, or consists of a honey, at least two fruit purees, and mint. In some embodiments, the honey is raw neem honey, the at least two fruit purees comprise kiwi puree, blackberry puree, and banana puree, and the mint is in leaf form. In some embodiments, the enhancing agent is any one or more of: matcha powder, charcoal powder, and maca powder.

### EXAMPLES

### Example 1: Sterilized Skin-Brightening Facial Masks

The food-grade components listed in Table 1 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-brightening facial mask cosmetic product.

TABLE 1

| Food-Grade Component | Amount | |
|---|---|---|
| lemon juice | 1 to 4 | ounces |
| Manuka honey | 4 to 12 | ounces |
| grated turmeric root | 1 to 4 | ounces |
| grated lemon peel | 0.5 to 4 | teaspoons |
| whole milk Greek yogurt | 50 to 200 | ounces |
| whole oat flour | 1 to 10 | ounces |

### Example 2: Sterilized Skin-Stimulating Facial Masks

The food-grade components listed in Table 2 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 2

| Food-Grade Component | Amount | |
|---|---|---|
| almond milk | 30 to 80 | ounces |
| raw cacao powder | 30 to 80 | ounces |
| coffee granules | 2 to 12 | ounces |
| dark maple syrup | 6 to 20 | ounces |
| extra virgin unrefined cold-pressed coconut oil | 2 to 12 | ounces |
| whole oat flour | 2 10 12 | ounces |

### Example 3: Sterilized Hydrating Facial Masks

The food-grade components listed in Table 3 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 3

| Food-Grade Component | Amount | |
|---|---|---|
| lime juice | 0.5 to 10 | ounces |
| raw honey | 2 to 12 | ounces |
| unrefined cold-pressed avocado oil | 2 to 12 | ounces |
| raw coconut water | 2 to 12 | ounces |
| avocado puree | 50 to 200 | ounces |

### Example 4: Sterilized Anti-Aging Facial Masks

The food-grade components listed in Table 4 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 4

| Food-Grade Component | Amount | |
|---|---|---|
| lemon juice | 2 to 12 | ounces |
| Manuka honey | 2 to 12 | ounces |
| extra virgin unrefined cold-pressed olive oil | 2 to 12 | ounces |
| whole milk Greek yogurt | 50 to 200 | ounces |
| vanilla bean powder | 0.5 to 4 | teaspoons |

### Example 5: Sterilized Balancing Facial Masks

The food-grade components listed in Table 5 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 5

| Food-Grade Component | Amount | |
|---|---|---|
| raw honey | 10 to 40 | ounces |
| pineapple pulp | 10 to 40 | ounces |
| papaya pulp | 10 to 40 | ounces |
| banana puree | 10 to 60 | ounces |

### Example 6: Sterilized Soothing Facial Masks

The food-grade components listed in Table 6 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 6

| Food-Grade Component | Amount | |
|---|---|---|
| cucumber pulp | 2 to 12 | ounces |
| aloe vera puree | 2. to 12 | ounces |
| whole milk Greek yogurt | 50 to 200 | ounces |
| kelp granules | 2. to 12 | ounces |

### Example 7: Sterilized Revitalizing Facial Masks

The food-grade components listed in Table 7 were combined in a power mixer until thoroughly blended to form about 4 L of cosmetic composition. 20 to 40-mL portions of the cosmetic composition were then placed into individual pouches and sealed. The sealed pouches were sterilized via high pressure process at about 600 MPa (about 87,000 psi) for about 3 minutes (i.e., 180 seconds) to form a sterilized skin-stimulating facial mask cosmetic product.

TABLE 7

| Food-Grade Component | Amount | |
|---|---|---|
| raw neem honey | 10 to 40 | ounces |
| kiwi puree | 10 to 40 | ounces |
| blackberry puree | 10 to 40 | ounces |
| banana puree | 10 to 60 | ounces |
| ground mint leaves | 0.5 to 4 | ounces |

While the present disclosure is capable of being embodied in various forms, the description herein of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

From the foregoing, it will be appreciated that specific embodiments of the invention have been described herein for purposes of illustration, but that various modifications may be made without deviating from the scope of the invention. Accordingly, the invention is not limited except as by the appended claims.

What is claimed is:

1. A cosmetic product comprising:
a pressure sterilized food grade mash, comprising:
a blend of least two or more of: a fruit juice, a fruit rind, a fruit pulp, a fruit puree, a vegetable puree, a succulent puree, a dairy product, flour, a natural oil, a natural syrup, honey, a plant based fat, herbs, herb extract, a nut or seed suspension, coconut, coconut water, kelp, a bean based powder, tea leaf, and a root based powder; and
an enhancing agent; and
a sealable container, wherein the container includes side seams and a sealable opening for holding the pressure sterilized food grade mash within the container under external hydrostatic pressure of up to 600 MPa.

2. The cosmetic product of claim 1, wherein:
the fruit juice comprises a citrus juice;
the fruit rind comprises a citrus peel; the fruit pulp comprises one or more of pineapple pulp, papaya pulp, mango pulp, melon pulp, and/or cucumber pulp;
the fruit puree comprises avocado puree, banana puree, kiwi puree, berry puree;
the succulent puree comprises aloe vera puree;
the dairy product comprises a yogurt;
the natural oil comprises a vegetable oil, coconut oil, avocado oil, and or olive oil;
the natural syrup comprises maple syrup, fruit syrup, or natural sugar based syrup;
the herbs comprise mint;
the nut or seed suspension comprises almond milk;
the bean based powder comprises vanilla powder, ground coffee, cocoa powder;
the tea leaf comprises machta powder; and
the root based powder comprises maca powder.

3. The cosmetic product of claim 1 wherein the enhancing agent further comprises one or more of matcha powder, charcoal powder, or maca powder.

4. The cosmetic product claim 1 wherein the container is flexible and water resistant.

5. The cosmetic product of claim 1 wherein the container comprises a food grade packaging material.

6. The cosmetic composition of any preceding claim, wherein the composition does not include an antimicrobial agent.

7. A method of making a sterilized cosmetic product, the method comprising:
blending a composition comprising at least two or more of a fruit juice, a fruit rind, a fruit pulp, a fruit puree, a vegetable puree, a succulent puree, a dairy product, flour, a natural oil, a natural syrup, honey, a plant based fat, herbs, herb extract, a nut or seed suspension, coconut, coconut water, kelp, a bean based powder, tea leaf, and/or a root based powder;
placing at least a portion of the composition in a flexible, water resistant, container;
sealing the container with the composition inside the container; and
pressure sterilizing the sealed container by surrounding the container in a fluid medium under pressure of between 200 MPa and 650 MPa.

8. The method of claim 7, wherein:
the fruit juice comprises a citris juice;
the fruit rind comprises a citris peel;
the fruit pulp comprises one or more of pineapple pulp, papaya pulp, mango pulp, melon pulp, and/or cucumber pulp;
the fruit puree comprises avocado puree, banana puree, kiwi puree, berry puree;
the succulent puree comprises aloe vera puree;
the dairy product comprises a yogurt;
the natural oil comprises a vegetable oil, coconut oil, avocado oil, and or olive oil;
the natural syrup comprises maple syrup, fruit syrup, or natural sugar based syrup;
the herbs comprise mint;
the nut or seed suspension comprises almond milk;
the bean based powder comprises vanilla powder, ground coffee, cocoa powder;
the tea leaf comprises machta powder; and
the root based powder comprises maca powder.

9. The method of claim 7 further comprising adding an enhancing agent to the composition after pressure sterilization.

10. The method of claim 7 further comprising adding an enhancing agent to the composition, wherein the enhancing agent comprises machta powder, charcoal powder, and/or maca powder.

11. A method of using a cosmetic product, the method comprising:
combining a sterilized cosmetic composition with an enhancing agent to form a ready-to-use cosmetic composition; and
applying the ready-to-use cosmetic composition to skin;
wherein the sterilized cosmetic composition comprises a blend of at least two or more of a fruit juice, a fruit rind, a fruit pulp, a fruit puree, a vegetable puree, a succulent puree, a dairy product, flour, a natural oil, a natural syrup, honey, a plant based fat, herbs, herb extract, a nut or seed suspension, coconut, coconut water, kelp, a bean based powder, tea leaf, and/or a root based powder; and
wherein the enhancing agent comprises one or more of a machta powder, charcoal powder, and/or maca powder.

12. The method of claim 11 wherein:
the fruit juice comprises a citris juice;
the fruit rind comprises a citris peel;
the fruit pulp comprises one or more of pineapple pulp, papaya pulp, mango pulp, melon pulp, and/or cucumber pulp;
the fruit puree comprises avocado puree, banana puree, kiwi puree, berry puree;
the succulent puree comprises aloe vera puree;
the dairy product comprises a yogurt;
the natural oil comprises a vegetable oil, coconut oil, avocado oil, and or olive oil;
the natural syrup comprises maple syrup, fruit syrup, or natural sugar based syrup;
the herbs comprise mint;
the nut or seed suspension comprises almond milk;
the bean based powder comprises vanilla powder, ground coffee, cocoa powder;
the tea leaf comprises machta powder; and
the root based powder comprises maca powder.

13. The method of claim 11 wherein the cosmetic composition is sterilized by placing the cosmetic composition in a flexible, water-resistant sealed container and surrounding the container in a fluid medium under pressure of between 200 MPa and 650 MPa for a period of between 10 seconds and 10 minutes.

14. The method of claim 13 wherein the enhancing agent is added to the cosmetic composition after sterilization of the cosmetic composition.

15. The method of claim 13 wherein an enhancing agent is omitted and the cosmetic composition is applied to the skin.
